(19) United States
(12) Patent Application Publication
(10) Pub. No.: US 2011/0301288 A1
(43) Pub. Date: Dec. 8, 2011

(54) THIOIC ACIDS AS BUILDING BLOCK FOR POLYTHIOESTERS

(75) Inventors: Mark Johannes Boerakker, Endhoven (NL); Aylvin Jorge Angelo Dias, Maastricht (NL); Ronny Mathieu Versteegen, Hegelsom (NL)

(73) Assignee: DSM IP ASSETS B.V., Heerlen (NL)

(21) Appl. No.: 13/133,217

(22) PCT Filed: Dec. 18, 2009
(86) PCT No.: PCT/EP09/67594

§ 371 Date: Aug. 22, 2011

(30) Foreign Application Priority Data
Dec. 19, 2008 (EP) 08022166.6

(51) Int. Cl. C09D 181/00; C08G 75/26; C07C 327/06; B82Y 30/00

(52) U.S. Cl. 524/600; 560/147; 562/26; 525/418; 528/373; 524/609; 524/599; 977/773; 977/788

(57) ABSTRACT

The present invention relates to thioic acids according to formula (II) suitable as building block for polythioesters; Wherein X is O, N or S and whereby R1 is chosen from (—CH₂—)₂, —CH₂—O—CH₂— or (—CH₂—)₃, R2 is preferably chosen from a biodegradable oligomer or polymer chosen from poly(lactide) (PLLA), polyglycolide (PGA), co-oligomers or copolymers of PLA and PGA (PLGA), poly(anhydrides), poly(trimethylenecarbonates), poly(orthoesters), poly(dioxanones), poly(ε-caprolactones) (PCL) or polyesteramides and n>1.

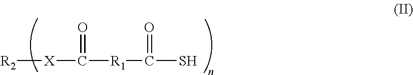

The present invention relates to new thioic acids as building blocks for polythioesters. The present invention further relates to a process for the preparation of thioic acids. The present invention also relates to polythioesters prepared from the thioic acids. The present invention further relates to medical devices comprising the polythioesters.

More in particular the invention relates to new thioic acids to produce polymers such as block copolymers or cross-linked networks that are suitable for medical applications. The invention provides polymers that may degrade completely when used in the human or animal body, minimizing residual components that may be toxic or otherwise undesirable.

Thioic acids are known in the prior art. From for example WO-A-2007028612 it is known that thioic acids can be used as building blocks for polythioesters. However the thioic acid containing compounds described so far are thioic acid compounds which comprise hydrophobic parts.

The polythioesters made from the thioic acid compounds comprising hydrophobic parts render degradation products with limited water solubility. The starting thioic acids will highly influence the polythioester properties, such as degradation or biodegradation but also the physical properties, the drug solubility and the release from polythioesters. For in vivo setting systems one needs to use water-soluble thioic acids which are not available at present. Finally, the hydrophobic nature limits their use for preparing hydrogel materials for biomedical applications.

The object of the present invention is to overcome the above mentioned disadvantages by providing a range of new thioic acids.

A still further object of the present invention is to provide the production of polythioesters from the new thioic acids.

It is still a further object of the present invention to provide polythioesters which hydrophilicity/hydrophobicity properties that can be tuned more easily. The ability to tune these properties would be of a great advantage in selecting a polythioester to release a specific drug. In this way the polythioesters can be tailored to reach optimal drug/polymer affinity and to obtain a desired release time.

The object of the present invention is achieved in providing new thioic acids according to formula II, suitable as building blocks for polythioesters,

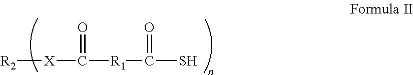

Wherein X is O, N or S and n>1;
R1 is chosen from (—CH2-)₂, —CH₂—O—CH₂— or (—CH₂—)₃ and
R2 is chosen from a low molecular weight compound having a Mw<1000 Da, oligomer or polymer.

If R2 is chosen from a low molecular weight compound having a Mw<1000 Da, it may be selected from the group consisting of ethyl, propyl, butyl, pentyl, ethyleneglycol, diethyleneglycol or triethyleneglycol.

Examples of R2 as oligomers or polymers are oligo-or respectively poly(vinyl alcohol) (PVA), oligo-or poly(ethylene oxide), poly(ethylene oxide)-co-poly(propylene oxide) block co-oligomers or copolymers, poloxamers, meroxapols, poloxamines, oligo-or poly(urethanes), oligo-or poly-esteramides, oligo-or polythioesteramides, oligo or poly((polyethyleneoxide)-co-poly(butyleneterephtalate)), oligo-or poly(vinyl pyrrolidone), oligo-or poly(ethyl oxazoline), carboxymethyl cellulose, hydroxyalkylated celluloses such as hydroxyethyl cellulose and methylhydroxypropyl cellulose, oligo-or poly(L-lactide) (PLLA), oligo-or poly (DL-lactide) (PDLLA), oligo-or polyglycolide (PGA), co-oligomers or copolymers of lactic acid and glycolic acid (PLGA), oligo-or poly(anhydrides), oligo-or poly(trimethylenecarbonates), oligo-or poly(orthoesters), oligo-or poly(dioxanones), oligo-or poly(ε-caprolactones) (PCL), oligo-or polyanhydrides and copolymers or co-oligomers or blends thereof.

Preferably R2 is chosen from a degradable oligomer or polymer. Examples of R2 as degradable or biodegradable oligomers or polymers are oligo-or poly(L-lactide) (PLLA), oligo-or poly (DL-lactide) (PDLLA), oligo-or polyglycolide (PGA), co-oligomers or copolymers of lactic acid and glycolic acid (PLGA), oligo-or poly(anhydrides), oligo-or poly(trimethylenecarbonates), oligo-or poly(orthoesters), oligo-or poly(dioxanones), oligo-or poly(β-caprolactones) (PCL), oligo-or polyanhydrides and copolymers or co-oligomers or blends of any of the above-mentioned polymers. In particularly preferred degradable oligomers or polymers are oligo-or PLLA, oligo-or PLGA, oligo-or PCL and copolymers of PCL and lactide or glycolide, oligo-or poly(urethanes), oligo-or poly(hydroxy acids), oligo-or polycarbonates, oligo-or polyaminocarbonates, oligo-or polyphosphazenes, oligo-or poly(propylene)fumarates, oligo-or polyesteramides, oligo-or polythioesteramides, oligo-or polyoxaesters, oligo-or poly(maleic acids), oligo-or polyacetals, oligo-or polyketals or natural polymers such as starch, polypeptides, polyhydroxyalkanoates, fibrin, chitin, chitosan, polysaccharides or carbohydrates such as polysucrose, hyaluronic acid, dextran and similar derivatives thereof, heparan sulfate, chondroitin sulfate, heparin, or alginate, and proteins such as gelatin, collagen, albumin, or ovalbumin, or co-oligomers or copolymers, or blends thereof.

More preferably R2 is chosen from oligo-or poly(lactide) (PLLA), oligo-or polyglycolide (PGA), co-oligomers or copolymers of lactic acid and glycolic acid, oligo-or poly(anhydrides), oligo-or poly(trimethylenecarbonates), oligo-or poly(orthoesters), oligo-or poly(dioxanones), oligo-or poly(ε-caprolactones) (PCL), oligo-or polyesteramide or oligo-or polythioesteramide.

R2 can however also be chosen from non-degradable oligomers or polymers such as oligo-or poly(vinyl alcohol) (PVA), oligo-or poly(ethylene oxide), poly(ethylene oxide)-co-poly(propylene oxide) block co-oligomers or copolymers, poloxamers, meroxapols, poloxamines, oligo-or poly(urethanes), oligo-or poly((polyethyleneoxide)-co-poly(butyleneterephtalate)), oligo-or poly(vinyl pyrrolidone), oligo-or poly(ethyl oxazoline), carboxymethyl cellulose, hydroxyalkylated celluloses such as hydroxyethyl cellulose and methylhydroxypropyl cellulose.

It is clear that the choice of R1, R2 and n in formula II will influence the properties of the resulting polythioester. n is preferably chosen from 2-8, more preferably from 2-6. If n=2 a lineair polymer may be formed. If n>2, a branched polymer may be formed.

Surprisingly it has been found that with the availability of the new thioic acids the hydrophilicity/hydrophobicity balance of the resulting polythioesters can be better tuned which results in better options to tune drug compatibility with the polythioester. In this way release characteristics, physical properties and degradation behaviour can be influenced. A further advantage is that the new thioic acids enable the preparation of waterborne formulations because of their more hydrophilic structure.

Finally the new thioic acids allow the preparation of hydrogels.

As used herein, the term “degradable” refers to a material having a molecular structure, which can decompose to smaller molecules. Such degradation or decomposition can be by various chemical mechanisms. For example, a degradable polymer can be hydrolytically degradable in which water reacts with the polymer to form two or more molecules from the polymer by chemical bonds in the molecule being hydrolyzed, thus producing smaller molecules. Many so-called degradable polymers are not completely degradable and have to be excreted, which puts strain on the kidneys and the renal system.

As used herein the term “biodegradable” refers to oligomers or polymers are materials that experience (accelerated) degradation by the action of biological agents produced by living organisms. Organic material can be degraded aerobically with oxygen or an-aerobically without oxygen.

As used herein, the term oligomer means a molecule of intermediate relative molecular mass, the structure of which essentially comprises a small plurality of units derived, actually or conceptually, from molecules of lower relative molecular mass. It is to be noted that a molecule is regarded as having an intermediate relative molecular mass if it has properties which do vary significantly with the removal of one or a few of the units. It is also to be noted that, if a part or the whole of the molecule has an intermediate relative molecular mass and essentially comprises a small plurality of units derived, actually or conceptually, from molecules of lower relative molecular mass, it may be described as oligomer, or by oligomeric used adjectivally. In general, oligomers have a molecular weight of more than 200 Da, such as more than 400, 800, 1000, 1200, 1500, 2000, 3000, 4000, or more than 8000 Da.

As used herein, the term polymer means a molecule of high relative molecular mass, the structure of which essentially comprises the multiple repetitions of units derived, actually or conceptually, from molecules of low relative molecular mass. Such polymers may include crosslinked networks, branched polymers and linear polymers. It is to be noted that in many cases, especially for synthetic polymers, a molecule can be regarded as having a high relative molecular mass if the addition or removal of one or a few of the units has a negligible effect on the molecular properties. This statement fails in the case of certain macromolecules for which the properties may be critically dependent on fine details of the molecular structure. It is also to be noted that, if a part or the whole of the molecule has a high relative molecular mass and essentially comprises the multiple repetition of units derived, actually or conceptually, from molecules of low relative molecular mass, it may be described as either polymer or by macromolecular or polymeric used adjectivally. In general, polymers have a molecular weight of more than 8000 Da, such as more than 10.000, 12.000, 15.000, 25.000, 40.000, 100.000 or more than 1.000.000 Da.

Another object of the present invention is to provide an easy process for the preparation of the thioic acids according to formula II.

The process according to the present invention is simplified in that it involves a single step with no side products, hence requiring no or little purification. In addition the process can be easily applied to various functional groups. This means that a broad range of new thioic acids become available so that new polythioesters can be designed.

The process according to the invention for the preparation of the new thioic acids according to formula II comprises the steps of:
a. reacting an alcohol, amine or thiol compound according to formula III with glutaric thioanhydride,succinic thioanhydride or diglycolic-thioanhydride
b. forming a compound of formula II

R₂Y)_{w} Formula III
Wherein R2 is a low molecular weight compound having a Mw<1000 Da, oligomer or polymer, Y is OH, NH2, R3NH, SH and w>1.
W is preferably chosen from 2-8, more preferably from 2-6.
R3 may be chosen from the same group as R2.
R2 and R3 may be the same or different, whereby R2 and R3 are as disclosed above.

The process according to the present invention provides the advantage of converting alcohols, amines or thiol functional molecules in one step into a thioic acid functional compound hereby broadening the group of starting molecules that can be converted into thioic acids via an easy route significantly. Moreover, the current route does not require dihydrogensulfide in the conversion of the functional group to a thioic acid group. Dihydrogensulfide is a toxic gas requiring extra safety measures when applied.

It is however also possible to prepare the thioic acids according to formula II by a process based on H₂S comprising the steps of:
a. reacting an alcohol, amine or thiol compound according to formula III with glutaric acid anhydride, succinic acid anhydride or diglycolic acid anhydride forming a compound of formula IV
b. converting compound of formula IV by the use of H₂S to thioic acid(s) according to formula II.

R₂Y)_{w} Formula III
Wherein R2 is a low molecular weight compound having a Mw<1000 Da, oligomer or polymer, Y is OH, NH2, R3NH, SH and w>1;
w is preferably chosen from 2-8, more preferably from 2-6;
R3 may be chosen from the same group as R2;
R2 and R3 may be the same or different;

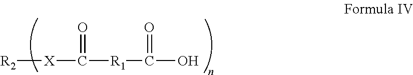

wherein X is O, N or S and n>1;
R1 is chosen from (—CH2—)₂, —CH₂—O—CH₂— or (—CH₂—)₃.

The invention further relates to polythioesters comprising the thioic acids according to formula II and/or thioic acids according to formula I

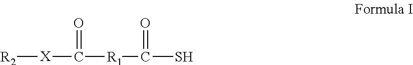

Wherein X is O, N or S and whereby
R1 is chosen from (—CH₂—)₂, —CH₂—O—CH₂— or (—CH₂—)₃; R2 is is chosen from a low molecular weight compound having a Mw<1000 Da, oligomer or polymer as disclosed in more specific detail above.

The thioic acids of formula I can be prepared according to the two above described processes with the proviso that w=1 and n=1.

The type of thioic acids, more specific the ratio of aromatic to aliphatic thioic acids, will influence the polythioester properties, such as water solubility and thus degradation or biodegradation, mechanical properties and hydrophilicity and drug solubility and release. When the ratio of aromatic to aliphatic thioic acids increases, the resulting polythioester is likely to be more hydrophobic, and have a higher mechanical strength. Conversely, when the ratio of aromatic to aliphatic thioic acids decreases, the resulting polythioester is likely to be more hydrophilic, and have a lower mechanical strength.

The polythioesters according to the present invention were found to be hydrolysable. Some of them were also found to be biodegradable. Depending on the choice of R2 in formula I or II, being an oligomer or polymer, degradability of the resulting polythioester may be influenced. For instance in case that R2 is a non-degradable triethyleneglycol divinyl ether (TEGDVE), lower degradation rates are achieved when compared to R2 being a degradable polymer such as poly(lactide-co-glycolide)1200di(4-pentenoate) (PLGDP) or poly(lactide-co-glycolide)2600tri(4-pentenoate) (PLGTP). If R2 is chosen being a hydrophobic component such as poly(ε-caprolactone) 2100di (4-pentenoate) (PCLDP) degradation over years is designed.

Particularly suited for applications in the human or animal body, such as in situ applications, is R2 being a polymer that can be degraded into degradation products without leaving any residual components. Such degradation products are then preferably non-toxic. The degradation products may be assimilated in the human or animal metabolism, or excreted.

Polythioesters according to the present invention may be prepared by mixing a compound Z comprising at least one ethylenically unsaturated group with the above described thioic acids of formula I and/or II.

Compound Z may be any suitable molecule having a carbon-carbon double bond. For example, the ethylenically unsaturated group may be selected from a group consisting of vinyl, alkyne, alkene, vinyl ether, vinyl sulphones, vinylphosphates, allyl, allylether, allylamine, acrylate, fumarate, maleate, itaconate, crotonate, citraconate, mesaconate, methacrylate, maleimide, isoprene, and norbornene and derivatives thereof such as esters and amides. Cyclic structures may also be used. Preferably component Z is a molecule having an ethylenically unsaturated group selected from a group consisting of alkene, vinyl ether, allyl, acrylate, fumarate, methacrylate, norbornene and derivatives thereof such as esters and amides.

Compound Z and the thioic acids of formula I and/or II are reacted by polymerisation. The thioester bonds may be formed employing a wide variety of mechanisms. Light and heat may be suitable or radical initiators but the reaction may also occur spontaneously. The skilled person knows that a wide variety of catalysts, thermal initiators, photo-initiators and stabilizers may be used to influence the speed and extent of the reaction. When used for in situ application in the human or animal body, a blue light or visible light source may be particularly advantageous. Alternatively a spontaneous reaction may be advantageous.

The temperature of the reaction provides a powerful tool to steer the speed and selectivity of the reaction. Also, the concentration of the reactants will determine the speed of the reaction and the properties of the polythioesters obtained.

Marvel and Kotch (J. Amer. Chem. Soc. 73, 1100-1102 (1951) and Kobayashi et al., Polymer Journal, 26, 49-59 (1994) and Polymer Journal, 25 507-520 (1993) describe a technology wherein components comprising ethylenically unsaturated groups are reacted with components comprising thioic acid groups.

Marvel and Kotch describe the preparation of polythioesters of the type [SCORCOS-R′]ₓ from a variety of dibasic chlorides and aliphatic dithiols or from the addition of dibasic thio acids (dithio adipic acid, dithiopimelic acid, dithio suberic acid, dithio-azelic acid, dithio-sebacic acid, dithio-terephtalic acid, dithio-isophtalic acid) to the non-conjugated diolefinbiallyl (1,5-hexadiene) using UV light.

Kobayashi et al., describe the reaction mechanism of the addition reaction of thiobenzoic acid to styrene or ethynylbenzene using UV light or radical initiators (AIBN) in Polymer Journal, 25, 501-520 (1993) and the polyaddition of 1,4-benzenedicarbothioic acid to 1,4-divinylbenzene or 1,4-diisopropenylbenzene in Polymer Journal, 26, 49-59 (1994). It is to be noted that the starting components used by Marvel and Kotch as well as those used by Kobayashi et al. do not qualify as oligomers or polymers. The starting thioic acids as disclosed in above two citations are hydrophobic thioic acids.

The properties of the polythioesters according to the present invention may further be influenced by the degree of cross-linking. Crosslinking may be achieved by choosing appropriate chain lengths of compound Z and of the thioic acids of formula I and/or II. Alternatively, the degree of cross-linking may be influenced by choosing an appropriate number of ethylenically unsaturated groups in compound Z. In another alternative the degree of cross-linking may be influenced by preventing the polymerization to go to completion, i.e. by preventing the highest degree of reaction to occur. Preferably, however, the reaction proceeds to the highest degree of reaction. A partial reaction may be especially desirable when it is required to have some residual reactive groups in the cross-linked matrix, for instance for modifications after cross-linking, such as attaching functional groups or covalent attachment to tissue or other biological material.

For the production of particularly useful linear polythioesters it may be advantageous that compound Z comprises a maximum of 2 ethylenically unsaturated groups and that the thioic acids of formula I and/or II comprise a maximum of 2 thioic acid groups.

For the production of particularly useful crosslinked polymers or networks, it is required that compound Z comprises at least 2 ethylenically unsaturated groups and that the thioic acids of formula I and/or II comprises at least 2 thioic acid groups and that the number of ethylenically unsaturated groups plus thioic acid groups is more than 4.

For the production of particularly strong crosslinked polymers or networks, it is required that compound Z comprises at least 3 ethylenically unsaturated groups and/or that the thioic acids of formula I and/or II comprise at least 3 thioic acid groups and that the number of ethylenically unsaturated groups plus thioic acid groups is more than 5.

For the production of particularly useful crosslinked polymers or networks, it is required that the composition comprising compound Z and thioic acids of formula I and/or II fulfils the boundary conditions for compositions for crosslinked polymers or networks as reported by Durand and Bruneau (D. Durand, C.-M. Bruneau, Makromol. Chem. 1982, 183, 1007-1020 and in D. Durand, C.-M. Bruneau, The British Polymer Journal, 1979, 11, 194-198; D. Durand, C.-M. Bruneau, The British Polymer Journal 1981, 13, 33-40; D. Durand, C.-M. Bruneau, Polymer, 1982, 23, 69-72; D. Durand, C.-M. Bruneau, Makromol. Chem., 1982, 183, 1021-1035; D. Durand, C.-M. Bruneau, Polymer, 1983, 24, 587-591).

Compound Z and R2 in the thioic acids of formula I and/or II may be based on the same oligomer or polymer, however, when they are based on different oligomers or polymers, the properties of the resulting polythioesters and distribution of active components such as drugs may be controlled more effectively and the reaction can be steered in a more controllable way. Compound Z and the thioic acids of formula I and/or II may both be degradable however one of the components may be partially degradable or non degradable. This is often utilised where an additional property as well as degradability is required.

In the case that degradable polythioesters are of importance compound

Z and/or R2 in the thioic acids of formula I and/or II can be both selected from oligo-or poly(lactide) (PLA), oligo-or polyglycolide (PGA), co-oligomers or copolymers of lactic acid and glycolic acid, oligo-or poly(anhydrides), oligo-or poly(trimethylenecarbonates), oligo-or poly(orthoesters), oligo-or poly(dioxanones), oligo-or poly(ε-caprolactones) (PCL), oligo-or poly(urethanes), oligo-or polyesteramides, oligo-or polythioesteramides, oligo-or polyanhydrides, oligo-or poly(hydroxy acids), oligo-or polycarbonates, oligo-or polyaminocarbonates, oligo-or polyphosphazenes, oligo-or poly(propylene)fumarates, oligo-or polyoxo-esters, oligo-or poly(maleic acids), oligo-or polyacetals, oligo-or polyketals. On the other hand compound Z and/or R2 in the thioic acids of formula I and/or II can also be both selected from natural polymers such as starch, polypeptides, oligo-or polyhydroxyalkanoates, fibrin, chitin, chitosan, polysaccharides or carbohydrates such as polysucrose, hyaluronic acid, dextran and similar derivatives thereof, heparan sulfate, chondroitin sulfate, heparin, or alginate, and proteins such as gelatin, collagen, albumin, or ovalbumin, or co-oligomers or copolymers, or blends thereof. In a particularly preferred embodiment compound Z and the R2 of the thioic acids of formula I and/or II are selected from oligo-or poly(lactide) (PLA), oligo-or poly(anhydrides), oligo-or polyesteramides, oligo-or poly(trimethylenecarbonates, oligo-or poly(dioxanones), oligo-or poly(ε-caprolactones) (PCL), oligo-or poly(lactide-co-glycolide) or co-oligomers or copolymers or blends thereof.

In the case that non-degradable polythioesters are required for an additional property like mechanical strength and/or non fouling properties, compound Z and/or R2 of the thioic acids of formula I and/or II may be selected from the group consisting of oligo-or poly(vinyl alcohol) (PVA), oligo-or poly(ethylene oxide), poly(ethylene oxide)-co-poly(propylene oxide) block co-oligomers or copolymers, oligo-or poloxamers, meroxapols, oligo-or polyesteramide, oligo-or poloxamines, oligo-or poly(urethanes), oligo-or poly((polyethyleneoxide)-co-poly(butyleneterephtalate)), oligo-or poly(vinyl pyrrolidone), oligo-or poly(ethyl oxazoline), carboxymethyl cellulose, hydroxyalkylated celluloses such as hydroxyethyl cellulose and methylhydroxypropyl cellulose.

Particularly good non fouling properties were achieved when using poly(ethylene oxide) as compound Z and R2 of the thioic acids of formula I and/or II. Particularly good amphiphilic behaviour was achieved when compound Z and R2 of the thioic acids of formula I and/or II were selected from the group consisting of poly(ethylene oxide)-co-poly(propylene oxide), poloxamers, poloxamines and meroxapols.

Particularly good mechanical strength was achieved when using polyurethanes as polymer in compound Z and as R2 of the thioic acids of formula I and/or II.

Particularly good hydrophilicity was achieved when compound Z and R2 of the thioic acids of formula I and/or II were selected from the group consisting of oligo-or polyethyleneoxide, oligo-or poly(vinyl pyrrolidone) and oligo-or poly(ethyl oxazoline).

Multiple compounds Z and/or thioic acids of formula I and/or II may also be used. Compound Z as well as R2 in the thioic acids of formula I and or II may consist of different oligomers or polymers. Compound Z and the thioic acids of formula I and/or II may vary in molecular weight depending upon which properties are desired for the resulting polythioester. More particularly, the molecular weight of compound Z and the thioic acids of formula I and/or II may range from about 28 Da to more than about 50000 Da. Prior to formation of the polythioester of the present invention, compound Z and the compound of formula III are derivatized to include thioic acid groups or ethylenically unsaturated groups such that they can participate in thioic-ene polymerisation. For use in in situ applications, the compound Z and the thioic acids of formula I and/or II are preferably of higher molecular weight.

The polythioesters of the present invention have the advantageous property that they can be degraded hydrolytically or enzymatically. When the compound Z and R2 in the thioic acids of formula I and/or II are degradable or biodegradable, a polythioester may be synthesized that can be degraded more completely with no residues left.

The invention further relates to medical devices comprising the thioic acids according to formula I or II and/or the polythioesters made thereof. Examples of medical devices are drug eluting balloons, stents, rods, screws, sutures, plates, pins, patches, vascular grafts, tissue engineering scaffolds or as materials for sutures. The thioic acids according to formula I or II and/or the polythioesters made thereof can however also be used as adhesives, anti-adhesives, sealants, meshes, sponges, hydrogels, waxes, films, cell delivery vehicles, coatings, polymer-based medicinal products or as matrices for the controlled delivery of drugs. The polythioesters are in particular useful as matrix polymer in controlled release systems or drug delivery systems such as in drug-eluting balloons or in other drug-eluting devices.

The invention further relates to microparticles comprising the polythioesters according to the present invention.

As used herein, microparticles include micro- and nanoscale particles or micelles which are typically composed of solid or semi-solid materials and which are capable of carrying an active agent. Typically, the average diameter of the microparticles given by the Fraunhofer theory in volume percent ranges from 10 nm to 1000 μm. The preferred average diameter depends on the intended use. For instance, in case the microparticles are intended for use as an injectable drug delivery system, in particular as an intravascular drug delivery system, an average diameter of up to 10 μm, in particular of 1 to 10 μm may be desired.

It is envisaged that nanoparticles typically have an average diameter of less than 1 μm, preferably less than 800 nm, and in particular less than 500 nm. These nanoparticles are very useful for intracellular purposes. For other applications smaller or larger average diameters may be required for instance a diameter in the range of 10-500 nm, 100-300 nm or 500-800 nm, hereinafter called micelles. In particular, the particle diameter as used herein is the diameter as determinable by a LST 230 Series Laser Diffraction Particle size analyzer (Beckman Coulter), making use of a UHMW-PE (0.02-0.04 μm) as a standard. Particle-size distributions are estimated from Fraunhofer diffraction data and given in volume (%). If the particles are too small or non analyzable by light scattering because of their optical properties then scanning electron microscopy (SEM) or transmission electron microscopy (TEM) can be used.

Several types of microparticle, nanoparticle or micelles structures can be prepared. These include substantially homogenous structures. In case that more than one active agent has to be released or in case that one or more functionalities are needed it is preferred that the microparticles, nanoparticles or micelles are provided with a structure comprising an inner core and an outer shell. A core/shell structure enables more multiple mode of action for example in drug delivery of incompatible compounds or in imaging. The shell can be applied after formation of the core using a spray drier. The core and the shell may comprise the same or different polymers such as for example the polythioesters according to the present invention, with different active agents. In this case it is possible to release the active agents at different rates. It is also possible that the active agent is only present in the core and that the shell is composed of the polythioesters according to the present invention.

In a further embodiment the microparticles, nanoparticles or micelles may comprise a core comprising the polythioesters according to the present invention and a shell comprising another material such as a magnetic or magnetisable material.

In still a further embodiment, the microparticles, nanoparticles or micelles may comprise a magnetic or magnetisable core and a shell comprising the polythioesters according to the present invention. Suitable magnetic or magnetisable materials are known in the art. Such microparticles, nanoparticles or micelles may be useful for the capability to be attracted by objects comprising metal, in particular steel, for instance an implanted object such as a graft or a stent. Such microparticles, nanoparticles or micelles may further be useful for purification or for analytical purposes.

In a still further embodiment, the micro-, nanoparticles or micelles may be imageable by a specific technique. Suitable imaging techniques are MRI, CT, X-ray. The imaging agent can be incorporated inside the particles or coupled onto their surface. Such particles may be useful to visualize how the particles migrate, for instance in the blood or in cells. A suitable imaging agent is for example gadolinium.

The microparticles, nanoparticles or micelles according to the present invention may carry one or more active agents. The active agent may be more or less homogeneously dispersed within the microparticles/nanoparticles core. The active agent may also be located within the microparticle, nanoparticle or micelle shell.

In particular, the active agent may be selected from the group of nutrients, pharmaceuticals, proteins and peptides, vaccines, genetic materials, (such as polynucleotides, oligonucleotides, plasmids, DNA and RNA), diagnostic agents, and imaging agents. The active agent such as an active pharmacologic ingredient (API), may demonstrate any kind of activity, depending on the intended use.

The active agent may be capable of stimulating or suppressing a biological response. The active agent may for example be chosen from growth factors (VEGF, FGF, MCP-1, PIGF, antibiotics (for instance penicillin's such as B-lactams, chloramphenicol), anti-inflammatory compounds, antithrombogenic compounds, anti-claudication drugs, anti-arrhythmic drugs, anti-atherosclerotic drugs, antihistamines, cancer drugs, vascular drugs, ophthalmic drugs, amino acids, vitamins, hormones, neurotransmitters, neurohormones, enzymes, signalling molecules and psychoactive medicaments.

Examples of specific active agents are neurological drugs (amphetamine, methylphenidate), alpha1 adrenoceptor antagonist (prazosin, terazosin, doxazosin, ketenserin, urapidil), alpha2 blockers (arginine, nitroglycerin), hypotensive (clonidine, methyldopa, moxonidine, hydralazine minoxidil), bradykinin, angiotensin receptor blockers (benazepril, captopril, cilazepril, enalapril, fosinopril, lisinopril, perindopril, quinapril, ramipril, trandolapril, zofenopril), angiotensin-1 blockers (candesartan, eprosartan, irbesartan, losartan, telmisartan, valsartan), endopeptidase (omapatrilate), beta2 agonists (acebutolol, atenolol, bisoprolol, celiprolol, esmodol, metoprolol, nebivolol, betaxolol), beta2 blockers (carvedilol, labetalol, oxprenolol, pindolol, propanolol) diuretic actives (chlortalidon, chlorothiazide, epitizide, hydrochlorthiazide, indapamide, amiloride, triamterene), calcium channel blockers (amlodipin, barnidipin, diltiazem, felodipin, isradipin, lacidipin, lercanidipin, nicardipin, nifedipin, nimodipin, nitrendipin, verapamil), anti arthymic active (amiodarone, solatol, diclofenac, enalapril, flecainide) or ciprofloxacin, latanoprost, flucloxacillin, rapamycin and analogues and limus derivatives, paclitaxel, taxol, cyclosporine, heparin, corticosteroids (triamcinolone acetonide, dexamethasone, fluocinolone acetonide), anti-angiogenic (iRNA, VEGF antagonists: bevacizumab, ranibizumab, pegaptanib), growth factor, zinc finger transcription factor, triclosan, insulin, salbutamol, oestrogen, norcantharidin, microlidil analogues, prostaglandins, statins, chondroitinase, diketopiperazines, macrocycli compounds, neuregulins, osteopontin, alkaloids, immuno suppressants, antibodies, avidin, biotin, clonazepam.

The active agent can be delivered for local delivery or as pre or post surgical therapies for the management of pain, osteomyelitis, osteosarcoma, joint infection, macular degeneration, diabetic eye, diabetes mellitus, psoriasis, ulcers, atherosclerosis, claudication, thrombosis viral infection, cancer or in the treatment of hernia.

The fields wherein microparticles, nanoparticles or micelles according to the present invention can be used include dermatology, vascular, orthopedics, ophthalmic, spinal, intestinal, pulmonary, nasal, or auricular. Besides in a pharmaceutical application, microparticles, nanoparticles or micelles according to the invention may inter alia be used in an agricultural application or in food applications. In particular, such microparticles, nanoparticles or micelles may comprise a pesticide, a plant-nutrient or food additives.

The invention will now be illustrated by the following examples without being limited thereto.

### General

NMR experiments were performed on a Varian Inova 300 spectrometer.

SEC analysis was performed using a Waters 515 HPLC pump, a Waters 410 Differential Refractometer and a Severn Analytical SA6503 Programmable Absorbance Detector equipped with a Waters styragel HR 2, 3 and 4 column at a flow rate of 1 ml/min using THF as the eluent. The system was calibrated using narrow polystyrene standards. UV irradiation was perfomed with a Photometrics Macam UV lamp (350 mW/cm²).

### Nomenclature

Polymers as described like PLGA-(50/50)-dithioic acid indicate a co-polymer of lactide and glycolide with a lactide/glycolide ratio of 50:50 initiated with diethyleneglycol and functionalized with thioic acid end groups.

Polymers as described like PCL-dodecanol-mono-ene indicate a polymer based on c-caprolactone which was initiated with 1-dodecanol and functionalized with an alkene end group.

Block-co-polymers as described like PCL-PLGA-PCL describe a tri-block copolymer of PCL and PLGA oligomers which were linked via thioester bonds.

### EXAMPLE 1

### Synthesis of Thioic Acid Using Succinic Anhydrides Starting from Alcohols

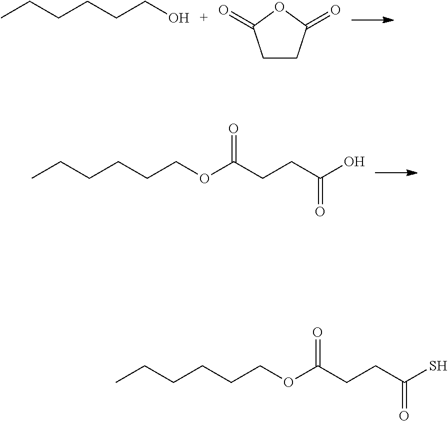

1-Hexanol (1.00 g; 9.8 mmol), pyridine (0.93 g; 11.7 mmol), and DMAP (0.24 g; 1.96 mmol) were dissolved in chloroform (20 mL). Succinic anhydride (1.18 g; 11.7 mmol) was added, and the reaction mixture was stirred at 70° C. under an argon atmosphere. After 18 hr, the reaction mixture was cooled to 20° C., chloroform (20 mL) was added, and this was washed twice with 1 M hydrochloric acid (20 mL) and water (10 mL). The organic layer was dried with sodium sulfate and evaporated to dryness, to yield the carboxylic acid as a colorless oil (1.64 g; 83%). CDl (0.441 g; 2.72 mmol) was dissolved in chloroform (4 mL) and to this solution was added drop wise a solution of the carboxylic acid (0.50 g; 2.47 mmol) in chloroform (2 mL). Subsequently, the reaction mixture was stirred at room temperature for 1 hr. Then, hydrogen sulfide was bubbled gently through the reaction mixture for 10 min, and the reaction mixture was stirred for an additional 90 min. Diethyl ether (15 mL) was added and the mixture was washed with 0.5 M sulfuric acid (10 mL). The organic layer was dried with sodium sulfate and evaporated to dryness, to yield a yellow oil (0.392 g; 73%).

The product was successfully characterized by ¹H-NMR and FT-IR.

¹H-NMR (CDCl₃): δ=0.88 (3H), 1.25 (6H), 1.62 (2H), 2.62 (2H), 2.97 (2H), 4.08 (2H) ppm

FT-IR: η=1713, 1731, 2556 cm⁻¹.

### EXAMPLE 2

### Synthesis of Thioic Acid Using Glutaric Anhydrides Starting from Alcohols

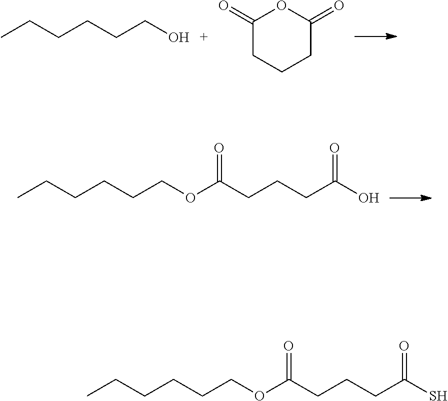

1-Hexanol (1.00 g; 9.8 mmol), pyridine (0.93 g; 11.7 mmol), and DMAP (0.24 g; 1.96 mmol) were dissolved in chloroform (20 mL). Glutaric anhydride (1.14 g; 11.7 mmol) was added, and the reaction mixture was stirred at 70° C. under an argon atmosphere. After 18 hr, the reaction mixture was cooled to 20° C., chloroform (20 mL) was added, and this was washed twice with 1 M hydrochloric acid (20 mL) and water (10 mL). The organic layer was dried with sodium sulfate and evaporated to dryness, to yield the carboxylic acid as a colorless oil (1.52 g; 72%).

CDl (0.412 g; 2.54 mmol) was dissolved in chloroform (4 mL) and to this solution was added drop wise a solution of the carboxylic acid (0.50 g; 2.31 mmol) in chloroform (2 mL). Subsequently, the reaction mixture was stirred at room temperature for 1 hr. Then, hydrogen sulfide was bubbled gently through the reaction mixture for 10 min, and the reaction mixture was stirred for an additional 90 min. Diethyl ether (15 mL) was added and the mixture was washed with 0.5 M sulfuric acid (10 mL). The organic layer was dried with sodium sulfate and evaporated to dryness, to yield yellow oil (0.345 g; 64%).

The product was successfully characterized by ¹H-NMR and FT-IR.

¹H-NMR (CDCl₃): δ=0.88 (3H), 1.25 (6H), 1.62 (2H), 1.97 (2H), 2.38 (2H), 2.71 (2H), 4.08 (2H), 4.45 (1H) ppm. FT-IR: η=1713, 1731, 2555 cm⁻¹.

### Example 3

### Synthesis of thioic Acid Using Glutaric(Thio)Anhydride Starting from an Alcohol

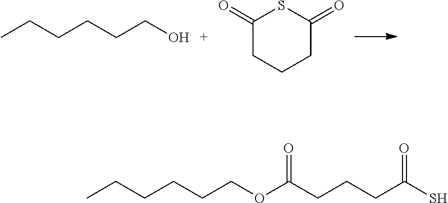

1-Hexanol (100 mg; 0.98 mmol) and DBU (179 mg; 1.17 mmol) were dissolved in chloroform (1.5 mL). Glutaric thioanhydride (127 mg; 0.98 mmol), dissolved in chloroform (1.5 mL) was added, and the reaction mixture was stirred at ambient temperature under an argon atmosphere for 2 hr. Chloroform (10 mL) was added and the mixture was washed with subsequently 0.1 M sulfuric acid (10 mL) and water (10 mL). The organic layer was dried with sodium sulfate and evaporated to dryness, to yield an orange oil (170 mg; 75%). The product was successfully characterized by ¹H-NMR (CDCl3): δ=0.88 (t, 3H), 1.30 (m, 6H), 1.60 (m, 2H), 1.95 (m, 2H), 2.35 (t, 2H), 2.68 (t, 2H) ppm.

Within 5 days at 20° C., the product was hardly degraded.

### Example 4

### Synthesis of Thioic Acid Using Glutaric(Thio)Anhydrides starting from a Primary Amine

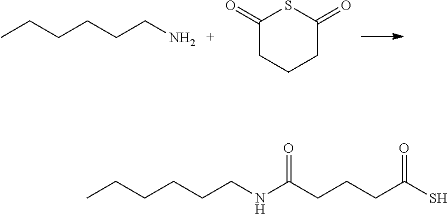

Glutaric thioanhydride (250 mg; 1.92 mmol), hexylamine (233 mg; 2.30 mmol) and triethylamine (233 mg; 2.30 mmol) were dissolved in chloroform (5 mL). The reaction mixture was stirred at ambient temperature under an argon atmosphere for 1 hr. Chloroform (5 mL) was added, and the mixture was washed with subsequently 0.5 M citric acid (10 mL) and water (10 mL). The organic layer was dried with magnesium sulfate and evaporated to dryness, to yield an off white waxy solid (340 mg; 77%). The product was successfully characterized by ¹H-NMR (CDCl₃): δ=0.88 (t, 3H), 1.30 (m, 6H), 1.47 (m, 2H), 1.98 (m, 2H), 2.22 (t, 2H), 2.72 (t, 2H), 3.22 (q, 2H) ppm.

The product was a little bit degraded during the work-up procedure.

Within 5 days at 20° C., the product was further degraded, mainly to the cyclic imide.

### EXAMPLE 5

### Synthesis of Thioic Acid Using Succinic(Thio)Anhydrides Starting from an Alcohol

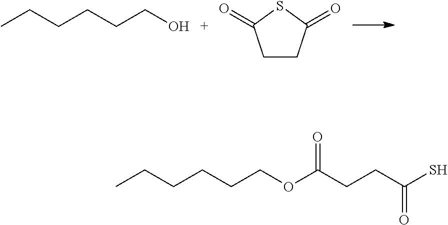

1-Hexanol (200 mg; 1.96 mmol) and Succinic thioanhydride (227 mg; 1.96 mmol) were dissolved in chloroform (4 mL). DBU (358 mg; 2.35 mmol), dissolved in chloroform (2 mL) was added, and the reaction mixture was stirred at ambient temperature under an argon atmosphere for 1 hr. Chloroform (20 mL) was added and the mixture was washed with subsequently 0.1 M sulfuric acid (20 mL) and water (20 mL). The organic layer was dried with sodium sulfate and evaporated to dryness, to yield yellow oil (390 mg; 91%). The product was successfully characterized by ¹H-NMR (CDCl₃): δ=0.88 (t, 3H), 1.32 (m, 6H), 1.61 (m, 2H), 2.62 (t, 2H), 2.98 (t, 2H), 4.05 (t, 2H) ppm.

The product was hardly degraded during the work-up procedure. Within 7 days at 20° C. or −20° C., the product did not further degradate.

### EXAMPLE 6

### Synthesis of Thioic Acid Using Succinic(Thio)Anhydrides Starting from a Primary Amine

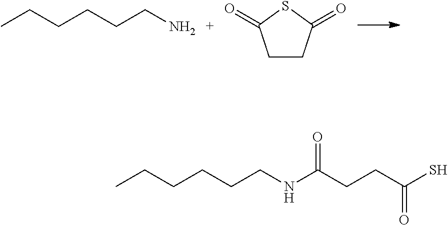

Succinic thioanhydride (250 mg; 2.15 mmol), hexylamine (261 mg; 2.58 mmol) and triethylamine (261 mg; 2.58 mmol) were dissolved in chloroform (6 mL). The reaction mixture was stirred at ambient temperature under an argon atmosphere for 30 min. Chloroform (5 mL) was added, and the mixture was washed with subsequently 0.5 M citric acid (10 mL) and water (10 mL). The organic layer was dried with sodium sulfate and evaporated to dryness, to yield a white solid (370 mg; 79%). The product was successfully characterized by ¹H-NMR (CDCl₃): δ=0.88 (t, 3H), 1.32 (m, 6H), 1.48 (m, 2H), 2.46 (t, 2H), 3.02 (t, 2H), 3.22 (q, 2H) ppm.

The product was slightly degraded during the work-up procedure. Within 7 days at −20° C., the product did not further degradate. Within 7 days at 20° C., the product was degraded for more than 50%, mainly to the cyclic imide.

### Example 7

### Synthesis of Thioic Acid Using Glutaric(Thio)Anhydrides Starting from a Secondary Amine

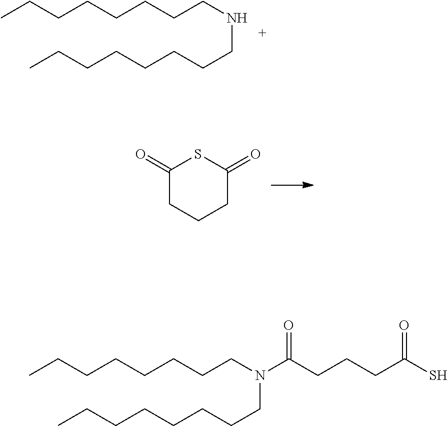

Glutaric thioanhydride (250 mg; 1.92 mmol), N,N-dioctylamine (464 mg; 1.92 mmol), and triethylamine (194 mg; 1.92 mmol) were dissolved in chloroform (4 mL). The reaction mixture was stirred at 20° C. under an argon atmosphere. After 45 min, chloroform (6 mL) was added, and this was washed four times with 0.1 M hydrochloric acid (8 mL). The organic layer was dried with sodium sulfate and evaporated to dryness, to yield a waxy solid (470 mg; 66%).

The product was successfully characterized by ¹H-NMR.

Within 6 days at 20° C., the product was hardly degraded.

¹H-NMR (CDCl₃): δ=0.88 (6H), 1.25 (20H), 1.50 (4H), 1.98 (2H), 2.38 (2H), 2.75 (2H), 3.18 (2H), 3.25 (2H) ppm

### EXAMPLE 8

### Synthesis of Thioic Acid Using Succinic(Thio)Anhydrides Starting from a Thiol

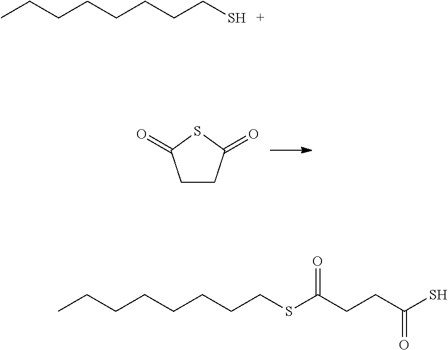

Succinic thioanhydride (58 mg; 0.50 mmol), 1-octanethiol (73 mg; 0.50 mmol), and triethylamine (51 mg; 0.50 mmol) were dissolved in chloroform (1 mL). The reaction mixture was stirred at 20° C. under an argon atmosphere. After 1 hr, chloroform (3 mL) was added, and this was washed twice with 0.1 M hydrochloric acid (2.5 mL). The organic layer was dried with sodium sulfate and evaporated to dryness, to yield a yellow oil (84 mg; 64%).

The product was successfully characterized by ¹H-NMR.

Within 4 days at 20° C., the product was degraded significantly.

¹H-NMR (CDCl₃): δ=0.88 (3H), 1.25 (10H), 1.58 (2H), 2.88 (4H), 2.99 (2H) ppm

### EXAMPLE 9

### Synthesis of Thioic Acid Using Glutaric(Thio)Anhydrides Starting from a Thiol

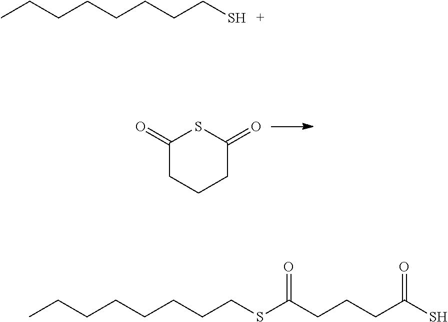

Glutaric thioanhydride (250 mg; 1.92 mmol), 1-octanethiol (280 mg; 1.92 mmol), and triethylamine (194 mg; 1.92 mmol) were dissolved in chloroform (4 mL). The reaction mixture was stirred at 20° C. under an argon atmosphere. After 1 hr, chloroform (6 mL) was added, and this was washed twice with 0.1 M hydrochloric acid (10 mL). The organic layer was dried with sodium sulfate and evaporated to dryness, to yield yellow oil (458 mg; 86%).

The product was successfully characterized by ¹H-NMR.

Within 4 days at 20° C., the product was hardly degraded.

¹H-NMR (CDCl₃): δ=0.88 (3H), 1.25 (10H), 1.57 (2H), 2.00 (2H), 2.61 (2H), 2.70 (2H), 2.87 (2H), 4.66 (1H) ppm.

### EXAMPLE 10

### Synthesis of Thioic Acid Using Glutaric(Thio)Anhydrides Starting from Polyethyleneglycol (PEG)

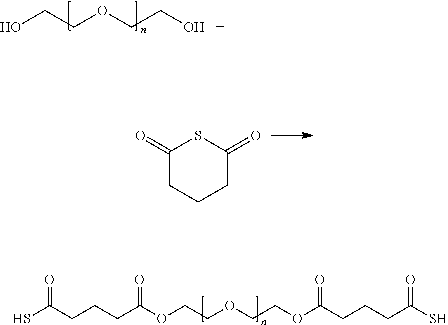

Poly ethyleneglycol (Mw=1000; 22.93 g; 22.93 mmol) and glutaric thioanhydride (5.97 g; 45.86 mmol) were dissolved in chloroform (200 mL). DBU (8.38 g; 55.04 mmol), dissolved in chloroform (50 mL) was added, and the reaction mixture was stirred at ambient temperature under an argon atmosphere for 3 hr. Chloroform (250 mL) was added, and the mixture was washed with subsequently 0.1 M sulfuric acid (400 mL) and water (400 mL). The organic layer was dried with sodium sulfate and evaporated to dryness, to yield brownish oil. The oil was co-evaporated twice with diethyl ether yielding a yellow waxy solid (27.2 g; 94%). The product was successfully characterized by ¹H-NMR (CDCl₃): □1.95 (m, 4H), 2.41 (1, 4H), 2.73 (t, 4H), 3.61 (m, 88H), 3.68 (t, 4.22 (t, 4H) ppm, ¹³C-NMR (CDCl₃): δ 20.28, 32.71, 44.62, 63.63, 69.07, 70.37, 70.58, 70.62, 172.61, 197.02 ppm and FT-IR: ν=2867, 1732 (C═O stretch), 1699 (C═O stretch) 1453, 1349, 1297, 1249, 1094 (C—O stretch), 1037, 948, 846 cm⁻¹.

### EXAMPLE 11

### Linear Polymers Based on PEG-1000 Dithioic Acid and PLGA

5.75 μL of dimethylethanolamine (DMEA) and 1.27 g of acetone were added to 0.9998 g (0.189 mmol) of a poly(D,L-lactide-co-glycolide) (Mₙ=5282 Da)dipentenoate with a lactide to glycolide ratio of 75:25. The mixture was allowed to mix overnight in the dark at room temperature by shaking (140 movements/min). Subsequently, 0.1615 g (0.0189 mmol) of a PEG-1000 dithioic acid (Mₙ=855 Da) was added. The mixture was shaken under the same conditions as mentioned above for 2 h. Finally, 0.015 mL of photoinitiator (Darocur 1173, 1 wt % w/w) was added and the mixture was shaken for an additional 30 minutes. The composition was applied onto a glass plate using a doctor blade (height=200 μm). The polymerization was performed using UV-light (Fusion F600 D-bulb, λ=320-380 nm, dose 15.2 J/cm²). The obtained material was placed in the oven at a temperature of 35° C. under vacuum overnight to remove any remaining volatile components to yield an off-white solid, which did not dissolve in water.

### EXAMPLE 12

### Synthesis of PCL-Dodecanol-Nono-Ene

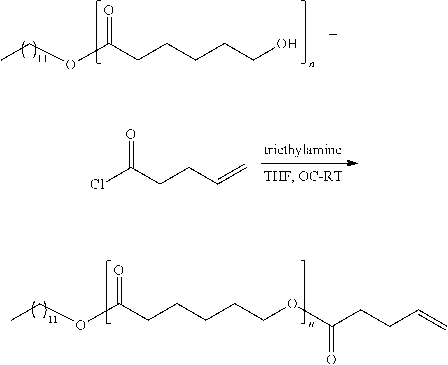

PCL-dodecanol-mono-ol (Mw=1300; 196.97 g, 0.151 mol) was dissolved in anhydrous THF (750 mL) in a 2L round bottomed flask. The mixture was cooled in an ice bath. Triethylamine (15,56 g, 0.154 mol) was added followed by drop wise addition of pentenoylchloride (17.98 g, 0.153 mol). The mixture was allowed to slowly warm to room temperature overnight. After cooling in an ice bath to enhance the crystallization of triethylamine.HCl, the cold solution was filtered over a Buchner funnel to remove triethylamine.HCl precipitate. All solvent was evaporated at the rotavapor. The crude material was dissolved in 500 mL of acetone. The acetone solution was dropwise added to 4000 mL stirred distilled water upon which the product precipitated. The solution was filtered over a Buchner funnel and rinsed with 500 mL distilled water. The material was dried under vacuum at 30° C. overnight. A off white waxy solid was obtained in a quantitative yield (210,67 g, 100%)

The product was successfully characterized by ¹H-NMR (CDCl₃): δ (ppm)=6.0-5.7 (0.9H, m), 5.2-5.0 (1.8H, m), 4.08-4.04 (21.5H, m), 2.45-2.28 (23.2H, m), 1.8-1.2 (80.5H, m) 0.88 (3.0H, t) ppm. SEC (relative to polystyrene standards): Mn=2205, Mw=2702, MP=2345, PDl: 1.225.

### EXAMPLE 13

### Synthesis of PLGA-(50/50)-Dithioic Acid

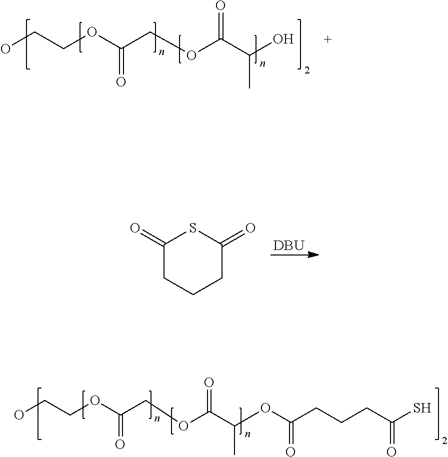

PLGA-(50/50)-diol (1) (Mw=1500; 12.18 g; 8.12 mmol) and glutaric thioanhydride (2.22 g; 17.05 mmol) were dissolved in chloroform (120 mL). DBU (2.97 g; 19.5 mmol), dissolved in chloroform (20 mL) was added, and the reaction mixture was stirred at ambient temperature under an argon atmosphere. After 1 hr, chloroform (140 mL) was added, and the mixture was washed with 0.1 M sulfuric acid (140 mL) and 0.05 M sulfuric acid (140 mL), subsequently. The organic layer was dried with sodium sulfate and evaporated to dryness, to yield orange oil (14.2 g; 99%). All actions of the workup procedure were performed under argon. The product was successfully characterized by ¹H-NMR (CDCl₃): δ5.21 (11H, m) 4.60-4.95 (m, 22H), 4.29 (4H, s), 3.68 (4H, m), 2.48 (4H, m), 2.00 (4H, m), 1.95 (33H, m) ppm. ¹³C-NMR (CDCl₃): δ 16.80, 20.26, 32.39, 32.37, 32.52, 37.93, 44.51, 60.39, 60.50, 60.91, 64.48, 68.78, 69.11, 69.27, 166.48, 169.44, 172.05, 197.0 ppm and FT-IR: ν=1996, 2951, 2561, 1747 (C=0 stretch), 1708 (C═O stretch) 1451, 1422, 1385, 1291, 1168, 1130, 1086, 962, 866, 946, 752 cm⁻¹.

### EXAMPLE 14

### Synthesis of Polycaprolactone-Monothioic Acid

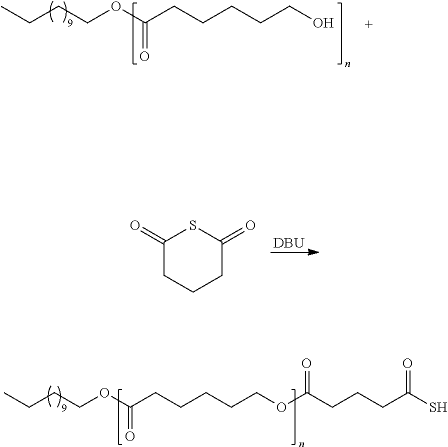

PCL-dodecanol-mono-ol (Mw=1200; 15.0 g; 12.5 mmol) and glutaric thioanhydride (1.73 g; 13.28 mmol) were dissolved in chloroform (140 mL). DBU (2.67 g; 17.54 mmol), dissolved in chloroform (20 mL) was added, and the reaction mixture was stirred at ambient temperature under an argon atmosphere. After 2 hr, chloroform (160 mL) was added, and the mixture was washed with 0.1 M sulfuric acid (160 mL) and 0.05 M sulfuric acid (160 mL), subsequently the organic layer was dried with sodium sulfate, concentrated to approximately 40 mL and precipitated in cold heptane (400 mL). Drying of the precipitate yielded a slightly orange powder (12.57 g; 76%). The product was successfully characterized by ¹H-NMR (CDCl₃): δ 4.02 (24H, t), 2.67 (2H, t), 2.35-2.26 (25H, m), 1.94 (2H, m), 1.23-1.62 (88H, m), 0.84 (3H, t) ppm. ¹³C-NMR (CDCl₃): δ14.21, 20.38, 22.77, 24.66, 25.62, 26.01, 28.39, 28.44, 28.72, 29.34, 29.43, 29.61, 29.66, 29.71, 31.99, 32.86, 34,21, 34.26, 44.70, 64.23, 64.46, 64.60, 172.78, 173.63, 197.03 ppm and FT-IR: ν=3442, 2942, 2875, 1722, 1471, 1419, 1397, 1365, 1293, 1238, 1171, 1108 ,1080, 1045 ,960, 933, 840, 769, 732 cm⁻¹. All actions of the workup procedure were performed under an argon atmosphere.

### EXAMPLE 15

### Synthesis of Polyethyleneglycol-Mono-Thioic Acid

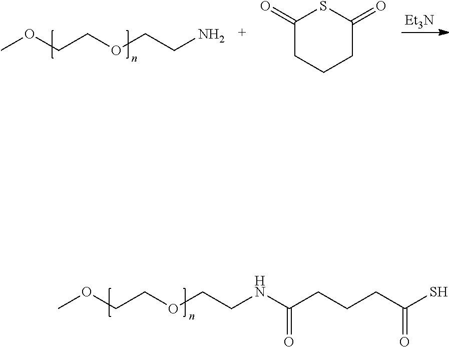

Glutaric thioanhydride (0.60 g; 4.60 mmol), PEG mono amine (Mw=2050; 9.51 g; 4.60 mmol) and triethylamine (0.56 g; 5.56 mmol) were dissolved in chloroform (100 mL). The reaction mixture was stirred at ambient temperature under an argon atmosphere. After 1.5 hr, chloroform (100 mL) was added, and the mixture was washed with 0.5 M citric acid (100 mL). The aqueous layer was extracted two more times with chloroform (50 mL). The combined organic layers were washed with water (100 mL), dried with magnesium sulfate, concentrated to approximately 35 mL and precipitated in cold diethylether (350 mL). Drying of the precipitate yielded a white powder (8.6 g; 85%). All actions of the workup procedure were performed under an argon atmosphere. The product was successfully characterized by ¹H-NMR (CDCl₃): δ 6.27 (1H, s), 3.82 (1H, t), 3.64 (180H, m), 3.55 (5H, m), 3.46 (3H, m), 3.39 (3H, s), 2.73 (2H, t), 2.25 (2H, m) ppm. ¹³C-NMR (CDCl₃): δ 21.06, 34.68, 39.20, 44.89, 59.05, 39.79, 70.23, 70.56, 71.93 ppm and FT-IR: ν=2883, 1653, 1544, 1466, 1360, 1341, 1280, 1241, 1147, 1101, 1060, 958, 947, 841 cm⁻¹.

### EXAMPLE 16

### Triblock PCL-PLGA-PCL

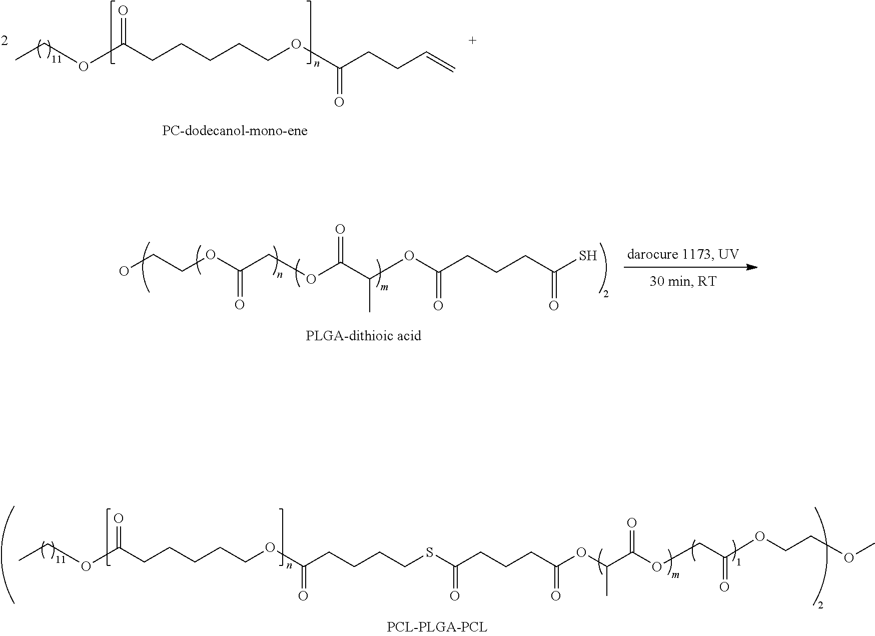

PLGA-dithioic acid (502.6 mg, 0.283 mmol) was added to PCL-dodecanol-mono-ene (776.0 mg, 0.565 mmol) the mixture was dissolved in 2 ml anhydrous THF. Darocure 1173 (20 mg, photoinitiator) was added. The mixture was irradiated with UV for 30 minutes. NMR was measured in CDCl₃ to confirm that a full conversion of the double bonds was obtained. A small portion of the reaction mixture was dried under a nitrogen flow and yielded a pale yellow product. The total yield was not determined. The material was successfully characterized by ¹H-NMR (CDCl₃): δ (ppm)=5.30-5.07 (10.4H, m); 4.79-4.66 (20.8H, m); 4.23 (4.0H, b), 4.10-3.90 (44.9H, m); 3.62 (10.7H, m); 2.85 (3,4H, t); 2.61 (3,5H, m); 2,45 (3,7H, t); 2.30-2.15 (43.8H, m); 2.1-1.9 (4.0H, m); 1.8 (7.3H, m); 1.64-1.15 (202.7H, m), 0.82, (6.3H, t) ppm; The sample contained a minor amount of the solvent THF. SEC (relative to polystyrene standards): Mn=6590, Mw=7543, MP=7102, PDl: 1.145.

A coating was prepared on a glass slide for determining the contact angle of the material, the contact angle of the material was 63 degree.

Water contact angles were measured with a TanTec cam-plus contact angle meter.

5 wt % polymer formulations were made in THF for the preparation of coatings. The polymer formulations were applied on glass slides with a thickness of 300 micrometers (Simex coating knife).

### EXAMPLE 17

### PEG-PCL-Di-Block Preparation

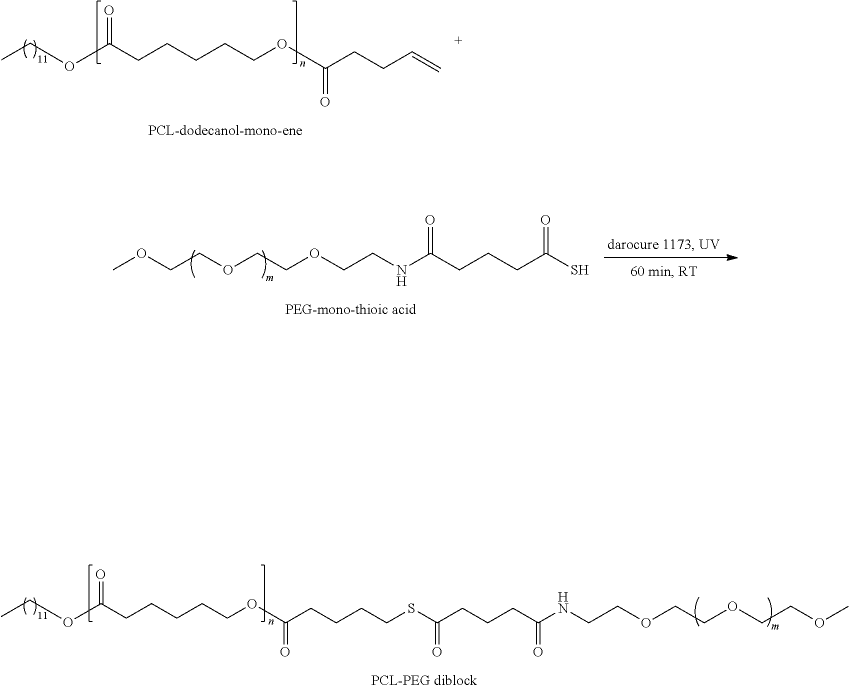

PEG-mono-thioic acid (1.0034 g, 0.46 mmol) was weighed in a 10 mL vial along with PCL-dodecanol-mono-ene (608.8 mg, 0.47 mmol). Darocure 1173 (9.8 mg) was added as the photo initiator. 2 mL anhydrous THF was added to dissolve the mixture. The mixture was UV irradiated for 30 minutes. NMR was measured in CDCl₃ to confirm that a full conversion of the double bonds was obtained. A small portion of the reaction mixture was dried under a nitrogen flow and yielded a pale yellow product. The total yield was not determined. The material was successfully characterized by ¹H-NMR (CDCl₃): δ (ppm)=6.20 (0.6H, b), 5.83-5.71 (0.2H, m), 5.10-4.97 (0.3H, m), 4.13-4.02 (43.4H, t), 3.90-3.50 (201H, m), 3.44-3.33 (1.9H, m), 3.32 (2.7H, s), 2.90-2.80 (1.4H, t), 2.63-2.55 (1.6H, t), 2.40-2.15 (24.1H, m), 2.00-1.90 (1.6H, m), 1.87-1.79 (5.8H, m), 1.70-1.50 (45.1H, m), 1.40-1.20 (76.6H, m), 0.84 (6.0H, t) ppm; SEC (relative to polystyrene standards): Mn=5117 Mw=5426, MP=5207, PDl: 1.060.

A coating was prepared on a glass slide for determining the contact angle of the material. The material dissolved during the measurement and was too hydrophilic to measure the contact angle.

### EXAMPLE 18

### Tri-Block PCL-PEG-PCL

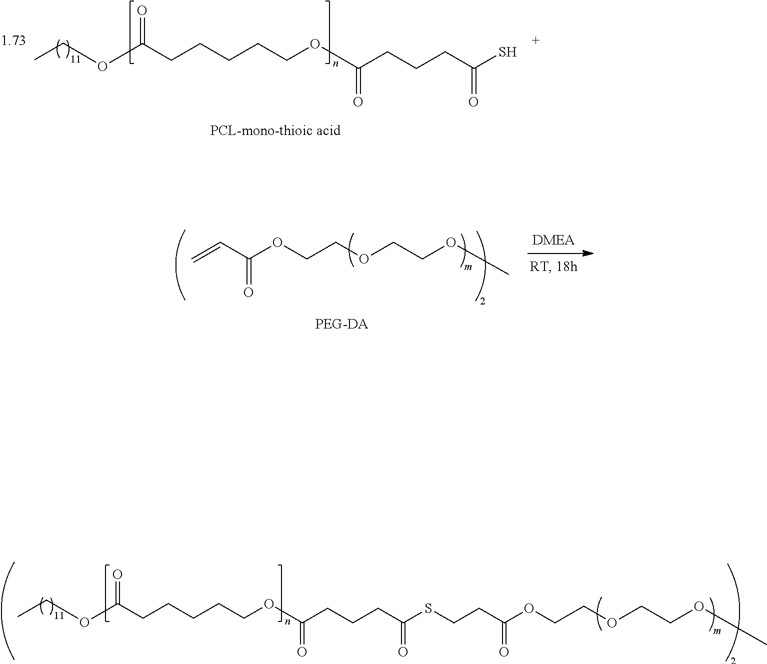

A stock solution of DMEA (123.1 mg, 1.38 mmol) and Irganox 1035 (12.1 mg, stabilizer) was prepared in acetone (12 mL). PCL-mono-thioic acid (800.7 mg, 0.56 mmol) was added in a brown sample vial followed by addition the addition of PEG-DA (171.7 mg, 0.25 mmol). The mixture was dissolved by the addition of the acetone DMEA stock solution (1 mL) and placed at room temperature overnight to allow the reaction to take place. The following day a small portion of the reaction mixture was dried under a nitrogen flow and yielded a pale yellow product. The total yield was not determined. The material was successfully characterized by ¹H-NMR (CDCl₃): δ (ppm)=4.33-4.22 (3.7H, m), 4.00-4.12 (46.6H, t), 3.72-3.61 (44.8H, m), 3.09 (3.2H, t), 3.00-2.84 (1.7H, m), 2.66-2.57 (6.7H, m), 2.40-2.25 (46.3H, m), 2.16 (0.4H, s), 2.01-1.90 (3.2H, m), 1.70-1.55 (89.5H, m), 1.44-1.22 (79.4H, m), 0.86 (6.0H, t) ppm; SEC (relative to polystyrene standards): Mn=6082, Mw=6502, MP=6009, PDl: 1.069.

A coating was prepared on a glass slide for determining the contact angle of the material. The material absorbed the water and was to hydrophilic to measure the contact angle. The material did not dissolve.

1. Thioic acids according to formula II; 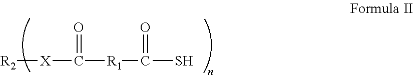
Wherein X is O, N or S and n>1;
R1 is chosen from (—CH₂—)₂, —CH₂—O—CH₂— or (—CH₂—)₃,
R2 is chosen from a low molecular weight compound having a Mw<1000 Da, an oligomer or a polymer.

2. Thioic acids according to claim 1 whereby R2 is chosen from a degradable oligomer or polymer.

3. Thioic acids according to claim 2 whereby R2 is chosen from a degradable oligomer or polymer selected from the group of oligo-or poly(lactide) (PLLA), oligo-or polyglycolide (PGA), fico-oligomers or copolymers of lactic acid and glycolic acid, oligo-or poly(anhydrides), oligo-or poly(trimethylenecarbonates), oligo-or poly(orthoesters), oligo-or poly(dioxanones), oligo-or poly(c-caprolactones) (PCL), oligo-or polyesteramides.

4. Thioic acids according to claim 1 whereby n=2.

5. Thioic acids according to claim 1 whereby n>2.

6. Process for the preparation of thioic acids according to claim 1 comprising the steps of
a. reacting an alcohol, amine or thiol compound according to formula Ill with glutaric thioanhydride, succinic thioanhydride or diglycolic thioanhydride
b. forming a compound of formula II
R₂Y)_{w} Formula III
Wherein R2 is chosen from a low molecular weight compound having a Mw<1000 Da, oligomer or polymer, Y is OH, NH2, R3NH, SH and w>1.
R3 may be chosen from the same group as R2.
R2 and R3 may be the same or different.

7. Process for the preparation of thioic acids according to claim 1 said process comprising the steps of
a. reacting an alcohol, amine or thiol compound according to formula III with glutaric acid, succinic anhydride, diglycolic anhydride forming a compound of formula IV
b. converting compound of formula IV by the use of H₂S to thioic acids according to formula II, 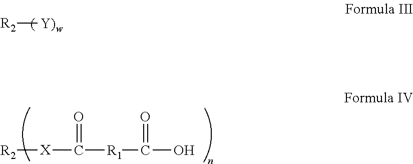
wherein X is chosen from O, N or S and w>1,
R1 is chosen from (—CH₂—)₂, —CH₂—O—CH₂— or (—CH₂—)₃;
R2 is chosen from a low molecular weight compound having a Mw<1000 Da, oligomer or polymer, Y is OH, NH2, R3NH or SH and R3 may be chosen from the same group as R2 whereby R2 and R3 may be the same or different.

8. Polythioesters comprising thioic acids according to claim 1.

9. Polythioesters comprising thioic acids according to formula I; 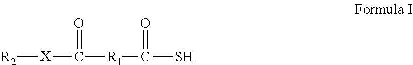
Wherein X is O, N or S and whereby
R1 is chosen from (—CH₂—)₂, —CH₂—O—CH₂— or (—CH₂—)₃; R2 is chosen from a low molecular weight compound having a Mw<1000 Da, oligomer or polymer.

10. Polythioesters according to claim 9 whereby R2 is chosen from polyethyleneglycol (PEG) or a degradable polymer or oligomer.

11. Polythioesters according to claim 9 whereby R2 is a degradable oligomer or polymer selected from the group of oligo-or poly(lactide) (PLLA), oligo-or polyglycolide (PGA), co-oligomers or copolymers of lactic acid and glycolic acid, oligo-or poly(anhydrides), oligo-or poly(trimethylenecarbonates), oligo-or poly(orthoesters), oligo-or poly(dioxanones), oligo-or poly(ε-caprolactones) (PCL), oligo-or polyesteramides.

12. Polythioesters according to claim 8 obtainable by mixing a compound Z comprising at least one ethylenically unsaturated group with thioic acids according to formula I 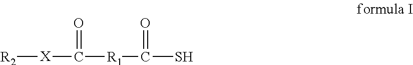
Wherein X is O, N or S and whereby
R1 is chosen from (—CH₂—)₂, —CH₂—13 CH₂— or (—CH₂—)₃ ; R2 is chosen from a low molecular weight compound having a Mw<1000 Da, oligomer or polymer.

13. Polythioesters according to claim 12 wherein the ethylenically unsaturated group of compound Z is chosen from the group consisting of alkene, vinyl ether, allyl, acrylate, fumarate, methacrylate or norbornene.

14. Medical device comprising polythioesters according to claim 8.

15. Use of the polythioesters according to claim 8 in drug delivery applications.

16. Micro-,nanoparticles or micelles comprising the thioic acids according to claim 1.

17. Coatings comprising the thioic acids according to claim 1.

18. Hydrogels comprising the thioic acids according to claim 1.
